(19) United States
(12) Patent Application Publication
(10) Pub. No.: US 2006/0257379 A1
(43) Pub. Date: Nov. 16, 2006

(54) Surgical system for the preparation of an implant and method for the preparation of an implant

(75) Inventors: Nicola Giordano, Villingen-Schwenningen (DE); Karl-Ernst Kienzle, Tuttlingen (DE); Andrea Weiler, Muehlheim (DE); Ulrich Fink, Tuttlingen (DE); Juergen Fritz, Dusslingen (DE)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/386,225

(22) Filed: Mar. 21, 2006

(30) Foreign Application Priority Data
Mar. 23, 2005 (DE) 10 2005 014 623.6

(51) Int. Cl. A61K 35/30; A61K 9/70

(52) U.S. Cl. 424/93.7; 424/443

(57) ABSTRACT

In order to improve a surgical system for the preparation of an implant for filling a defect on a bone and/or a cartilage of a human or animal body formed as a result of a trauma and/or degeneration, so that the defect can be filled with the implant with as precise a fit as possible, it is proposed that the system enables a provided defect data file to be processed, which contains data in particular for the description of the shape, area and/or volume of the defect, and that the system enables the implant corresponding to the defect in shape and size to be prepared from at least one provided implant material using the defect data file. In addition, a method for filling a defect on a bone and/or cartilage of a human or animal body present as a result of a trauma and/or degeneration is proposed.

The present disclosure relates to the subject matter disclosed in German patent application 10 2005 014 623.6 of Mar. 23, 2005, which is incorporated herein by reference in its entirety and for all purposes.

### BACKGROUND OF THE INVENTION

The present invention relates to a surgical system for the preparation of an implant for filling a defect on a bone and/or a cartilage of a human or animal body formed as a result of a trauma and/or degeneration.

In addition, the present invention relates to a method for the preparation of an implant for filling a defect on a bone and/or a cartilage of a human or animal body present as a result of a trauma and/or degeneration.

It is known to treat cartilage defects on joints, in particular traumatic cartilage defects in the knee with an area of 4 to 15 cm², preferably by means of a biological reconstruction. A possible type of treatment, in particular in the case of smaller defects with defect areas of less than 4 cm², consists of filling the defect with a sponge-like support material. Preferably, with this method of treatment, before the implantation, i.e. before the insertion of a replacement cartilage comprising the support material, the subchondral plate of the cartilage defect is lightly perforated in a targeted manner, which is referred to as “microfracturing”. As a result of the microfracturing of the subchondral plate, autologous cartilage cells or cells from the fat marrow tissue of the spongiosa lying below the defect bleed into the support material. The cells from the fat marrow tissue can be at least partially differentiated into cartilage cells.

Autologous chondrocyte transplantation (ACT) forms an alternative method of treatment. With this method of reconstruction cartilage cells, preferably removed from the patient to be treated, are cultured ex vivo, propagated, and then incorporated into the sponge-like support material, which forms at least a part of a replacement body to be inserted into the defect, which is also referred to as “inoculation” of the support material with the cartilage cells. The replacement body comprising the inoculated support material is then inserted into the body of the patient to fill the defect. The replacement body comprising the inoculated support material is usually referred to as a “transplant”.

The term “implant” is used below for a replacement body comprising a support material that is not inoculated prior to insertion, an implant in the proper sense, and also for a replacement body comprising an inoculated support material prior to insertion (“transplant”).

It is a prerequisite for both procedures to match the support materials intra-operatively optimally to the individual geometry of the actual defect. Thus, in particular a gap between the support material and a remaining healthy cartilage collar is absolutely unacceptable. While it is to some extent readily possible in an open operation technique to identify the size and shape of the defect precisely, it is largely impossible to determine the size and shape of the defect in an arthroscopic operation technique.

Therefore, it would be desirable to improve a system and a method of the above-described type so that the defect can be filled with the implant with as precise a fit as possible.

### SUMMARY OF THE INVENTION

The present invention proposes an improved surgical system of the above-described type, wherein the system enables a provided defect data file to be processed, which contains data in particular for description of the shape, area, height and/or volume of the defect, and that the system enables the implant corresponding to the defect in shape and size to be prepared from at least one provided implant material using the defect data file.

The implant can be prepared in a simple manner with such a surgical system according to the invention. The defect data file contains all the data necessary for this. For example, the system enables the shape of the defect, its area and/or its volume to be determined from the defect data file and further processed to prepare an implant from the provided implant material, which corresponds in shape and size to the defect and completely fills this after implantation. In this way, a gap, e.g. between a cartilage replacement implant and a healthy cartilage collar, can be prevented from forming. Such gaps can only be regenerated with great difficulty by the body and can form the starting point of growing defects.

It is advantageous if the implant is a cartilage replacement implant for autologous chondrocyte transplantation (ACT) and if the defect forming a cartilage defect can be filled with the cartilage replacement implant. Therefore, it is possible with the system to prepare a cartilage replacement implant, with which a cartilage defect in a human or animal body can be treated.

It is favourable if the implant material is a support material suitable for inoculation with cartilage cells, if the cartilage replacement implant comprises a support for the cartilage cells, and if the support is prepared from the support material. Such a support material allows the cartilage replacement implant to be inoculated with cartilage cells before implantation and then implanted. As a result, a regeneration of the cartilage defect can be significantly improved.

To facilitate the inoculation of the support material with cartilage cells, it is advantageous if the support material is a nonwoven material. For example, the nonwoven material can be a collagen nonwoven. The support material preferably has a thickness corresponding to a thickness or height of the defect, e.g. in the case of a cartilage defect a thickness in the range of 1 to 5 mm.

It would be conceivable in principle to implant the cartilage replacement implant without inoculating with cartilage cells, as has already been described above. However, advantageously the support can be inoculated with cartilage cells. In this way, cartilage cells can be incorporated in a targeted manner into the support prior to implantation.

According to a preferred embodiment of the invention it can be provided that the support can be inoculated with cartilage cells, which are cartilage cells from the body itself propagated in the laboratory, which were removed from the human or animal body before insertion of the cartilage replacement implant. By using cartilage cells from the body itself any rejection reaction of the cartilage replacement implant can be minimised or even completely prevented.

It is advantageous if the defect volume of the cartilage defect can be determined from the defect data file, and if a provided implant volume of cartilage cells is sufficient to adequately inoculate the support with cartilage cells for implantation of the cartilage replacement implant. If the defect volume is known, the propagation of cartilage cells can be optimised, since it is not necessary to culture more cartilage cells than are required to fill the defect volume. Thus, an upper limit for an implant volume to be provided can be specified.

It is advantageous if the provided support material has cavities to receive cartilage cells, if the cavities of the support have a cavity volume, and if a provided implantation volume corresponds to the cavity volume or approximately to the cavity volume of the support. As a result, the propagation of the cartilage cells can be further optimised, since the cavity volume of the support is generally smaller than the defect volume. As a result, the costly propagation of cartilage cells can be limited to the required degree.

According to a preferred embodiment of the invention, a data processing unit an be provided for processing the defect data file with the data processing unit to form a preparation data file, wherein the preparation data file contains data in particular for description of the shape, area, height and/or volume of the implant to be prepared. For example, the preparation data file can be used to machine the implant material with a work tool or a machining device in the desired way, i.e. to configure or prepare an implant corresponding to the defect.

It is beneficial if a surgical instrument is provided for opening a minimally invasive access into the human or animal body to determine the defect data file. Therefore, it is no longer necessary to fully open a joint, for example, instead the defect data file can be determined using a minimally invasive or arthroscopic access.

As mentioned above, the determination of the defect size and with it the defect data file is relatively simple, if an open operating technique is applied. Since in the case of autologous chondrocyte transplantation, for example, cartilage cells are removed arthroscopically, it would be desirable to also determine the defect data file arthroscopically, in particular during this procedure. Studies have shown, however, that even experienced arthroscopists incorrectly estimate the defect area by up to a factor of 2, even when using probe hooks with a measuring function, as a result of the distortion of the image during the arthroscopy. Therefore, it is advantageous if a navigation system is provided with a detection device, that a measuring instrument is provided, on which a reference element detectable by the detection device of the navigation system can be arranged for determination of the position and orientation of the measuring instrument in space, and that the defect can be measured with the measuring instrument for determination of the defect data file and at the same time the position and orientation of the measuring instrument in space can be determined with the navigation system. For determination of the measurement data file, a surgeon no longer has to estimate, but can measure the defect with high precision using the measuring instrument. This is possible through the navigation of the measuring instrument, since in this way its position and orientation are always known exactly during the measurement. Consequently, any distorted image of the defect possibly perceived by the arthroscopist becomes insignificant, since the defect data file contains the actual geometric data of the defect.

It is beneficial if the measuring instrument can be inserted into the human or animal body through a minimally invasive access. As a result, it is no longer necessary to apply an open operating technique, instead the measuring instrument can be inserted into the body just after a minimally invasive access is opened, e.g. for the removal of autologous cartilage cells. For this purpose, it is advantageously configured in the form of an endoscopic instrument.

According to a preferred embodiment of the invention, it can be provided that the measuring instrument has a probe tip, and that an edge or a boundary of the defect can be palpated with the probe tip for measurement of the defect. A surgeon can probe the edge or boundary of the defect with the probe tip, wherein position data of the probe tip are simultaneously determined through the detection device. The shape, size and/or volume of the defect, for example, can then be determined from the defect data file determined in this way.

It is beneficial if the measuring instrument is configured such that the defect can be measured without contact. This has the advantage that the defect is not enlarged further through the measurement. For example, the measuring instrument can use electromagnetic radiation, in particular visible light, or ultrasound to measure the defect.

According to a further preferred embodiment of the invention, it can be provided that the measuring instrument has an optical imaging unit, and that at least one defect image of the defect can be recorded with the imaging unit. All the data relevant to the defect can be determined from the defect image recorded through navigation, i.e. in particular the shape, area, height and/or volume of the defect. Moreover, the imaging unit has the advantage that the defect can be measured without contact. If, in addition, the body of the patient is also navigated, real spatial data of the defect can thus be acquired via the optical imaging unit, also in real time, and can be further processed with a data processing device. Moreover, it can thus also be established in particular whether the defect affects not only the cartilage, but also the subchondral plate located under it or even the bone. If this is the case, then the defect on the subchondral plate and/or on the bone can be filled by bone chips or bone replacement material so as not to put at risk the success of the treatment of the cartilage defect.

It is beneficial if an image data file of the at least one defect image can be provided by the imaging unit, and if the image data file can be processed to form the defect data file. A data processing unit is preferably provided for processing the image data file to form the defect data file. Thus, it is possible, for example, to generate the defect data file and, if desired or necessary, to use the defect data file directly close to time in order to prepare the implant during the surgical procedure.

So that the defect can also be measured without any additional illumination, it is advantageous if at least one infrared sensor is provided as imaging unit. With the at least one infrared sensor, the defect can be detected without contact and an image data file can be determined.

In a preferred embodiment of the invention, a work tool can be provided for machining the implant material, and the implant material can be machined with the work tool using the defect data file. In this way it is possible to prepare the implant with the work tool in the desired way, since all the data necessary for preparation of the implant are contained in the defect data file, in particular shape, size, area, height and/or volume of the defect.

In principle, it would be conceivable that the work tool is a part of a machining device, which prepares the implant in a controlled manner according to the given information of the defect data file. However, it is advantageous if the work tool has a reference element detectable by the detection device, and if the implant material can be machined with the work tool with simultaneous detection of the position and orientation of the work tool. The implant can be prepared manually with such a work tool using the defect data file, e.g. by drawing a contour on an implant material or by cutting it out with a cutting tool, in particular scissors, a punch or a laser. For example, a path of movement to be covered with the work tool, i.e. a desired curve, can be displayed on a display device, e.g. a screen, for this purpose. At the same time, an actual path of movement of the work tool can be superposed on the desired curve, so that an operator always knows exactly whether he/she is machining the implant material in the desired manner.

It is beneficial if the work tool comprises a marking tool for transferring the defect data file to the implant material. For example, the implant material can thus be marked in the desired manner in order to then machine it. In particular, a support material, e.g. a nonwoven material, can be marked in this way corresponding with an area of the defect known from the defect data file and cut out after marking.

In addition, it can be advantageous if the work tool comprises a cutting device for cutting the support out of the implant material using the defect data file. For example, with the work tool either the defect data file can be transferred to the implant material or it can also be cut out directly with the cutting device.

To enable the implant to be prepared fully automatically, it is beneficial if a machining device is provided for machining the implant material, if the defect data file is transferable to the machining device, and if the implant material can be machined with the machining device. The machining device can be actuated using the defect data file, but alternatively it would also be conceivable that the machining device additionally bears a reference element, which can be monitored with the navigation system, and in this way the implant material is machined through navigation.

According to a preferred embodiment of the invention, it can additionally be provided that the machining device comprises the work tool. Different work tools can be provided for different implant materials, e.g. punching or cutting tools, in particular knife-like tools, or also radiation cutting tools such as lasers or similar.

In addition, it can be provided that the system comprises the implant. Thus, the implant is thus a part of the system for the treatment of a defect on a human or animal body.

Moreover, the present invention proposes an improved method of the above-described type which comprises the steps:
providing at least one implant material;
providing a defect data file, which contains data for description in particular of the shape, area, height and/or volume of the defect, and
preparing the implant from the implant material corresponding to the defect in shape and size using the defect data file.

With the method according to the invention, an implant, with which the defect can be filled at least partially, preferably completely, can be provided in a simple manner. Moreover, the method does not have to be performed by a doctor, since by the creation of the defect data file the implant can be prepared independently of a surgical procedure and can be made ready for insertion into the human or animal body.

It is particularly advantageous if the defect is a cartilage defect, if the implant is a cartilage replacement implant for autologous chondrocyte transplantation (ACT), and if the cartilage defect is filled with the cartilage replacement implant. A cartilage replacement implant can be prepared for the treatment of a cartilage defect on a knee joint, for example, in a simple manner with the method according to the invention. In contrast to known operating techniques, it is now possible to prepare a cartilage replacement implant for a precise fit, so that no gap can form between the implanted cartilage replacement implant and a remaining cartilage edge.

It is beneficial if a support material suitable for inoculation with cartilage cells is provided as implant material, if the cartilage replacement implant comprises a support for the cartilage cells, and if the support is prepared from the support material. It is possible in this way in particular to incorporate cartilage cells from the body itself into a support material and provide them for implantation. Such implants exhibit significantly smaller or even no rejection reactions. Because of the optimised fit of the implant, the cartilage cells from the body itself can grow in particularly well. The implant material can be homogeneous or can consist of different layers, which, however, do not all have to be suitable for inoculation with cartilage cells. For example, a covering or protective layer for the support inoculated with cartilage cells can also be provided.

To enable the cartilage replacement implant to absorb cartilage cells particularly well, it is advantageous if a nonwoven material is used as support material. A nonwoven material is light, has sufficient cavities and is, moreover, particularly easy to work with.

In principle, it would be conceivable to insert the prepared support directly into the cartilage defect, in particular without inoculating it with cartilage cells before implantation or insertion. With this operating technique, the subchondral plate of the cartilage defect is preferably microfractured, as described above, so that autologous cartilage cells, in particular from the fat marrow tissue of the spongiosa lying below it, which play a decisive role in cartilage regeneration, bleed into the support material. However, it is advantageous if the support is inoculated with cartilage cells after preparation and before implantation. Thus, it is possible to incorporate cartilage cells into the support in a targeted manner so that cell growth can be assured as reliably and quickly as possible directly after implantation.

The ingrowth of the cartilage replacement implant is additionally improved and possible rejection reactions of the body reduced or even excluded altogether if cartilage cells are removed from the human or animal body before implantation of the cartilage replacement implant, if the removed cartilage cells from the body itself are propagated in the laboratory, and if the propagated cartilage cells from the body itself are provided for inoculation of the support.

To ensure that only the amount of cartilage cells that is absolutely necessary is provided, which in particular minimises the production expenditure and the costs associated with it, it is beneficial if the defect volume of the cartilage defect is determined from the defect data file, and if an implant volume of cartilage cells is provided, wherein the implantation volume is sufficient to adequately inoculate the support with cartilage cells for an implantation of the cartilage replacement implant. Therefore, the number of cartilage cells provided is only that actually required for inoculation of the cartilage replacement implant. This additionally has the advantage that in the case of small defects, an implantation of the cartilage replacement implant can occur after only a much shorter period calculated from the removal of the cartilage cells from the body itself.

The implantation volume of cartilage cells to be provided can be further reduced if the provided support material has cavities to receive cartilage cells, if the cavities of the support have a cavity volume, and if an implantation volume is provided, which corresponds to the cavity volume or approximately to the cavity volume of the support. When the support material is known, the cavity volume of the implant can be determined in a simple manner. A volume corresponding to the cavity volume or approximately to the cavity volume of the support is then sufficient as implantation volume.

To enable the implant to be prepared in a simple manner, it is beneficial if the defect data file is processed with a data processing unit to form a preparation data file, wherein the preparation data file contains data for the description in particular of the shape, area and/or volume of the implant to be prepared. The preparation data file can be generated particularly quickly with the data processing unit, so that a cartilage replacement implant can be prepared directly after determination of the defect data file, e.g. still during a surgical procedure.

A minimally invasive access into the human or animal body is advantageously opened to determine the defect data file. Trauma during the operation can be minimised through the minimally invasive access. Moreover, the risk of infection is substantially reduced.

It is advantageous if an edge of the defect is prepared before determination of the defect data file. By preparing the edge of the defect, in particular removing loose fragments not connected to the subchondral bone plate or cutting the defect edge, a gap can be prevented from forming between the implant and the defect edge. Thus, the edge of the defect, which the implant adjoins after insertion, is determined exactly, so that this can be prepared to optimally fill the defect.

According to a preferred variant of the method according to the invention, it can be provided that a measuring instrument is provided, on which a reference element detectable by a detection device of a navigation system can be arranged for determination of the position and orientation of the measuring instrument in space, and that the defect is measured with the measuring instrument for determination of the defect data file and at the same time the position and orientation of the measuring instrument is determined in space. By using such a measuring instrument, the size of the defect can be determined particularly accurately, even spatial coordinates of the defect can be specified in the respective reference system, for example, in particular also in real time. This can prevent, for example, too few cartilage cells from being provided for inoculation of a cartilage replacement implant. While this could be prevented by providing a significantly larger implantation volume of cartilage cells than actually required, this also substantially increases the costs for a procedure. Moreover, the quality of the cells decreases as the passaging increases, i.e. the smaller the number of required passages, the better the quality of the cells. Moreover, the precise measurement also allows the formation of a gap between the implant and a boundary of the defect to be prevented after implantation of the implant.

It is particularly advantageous if the measuring instrument is inserted into the human or animal body through the minimally invasive access. Then the defect can not only be measured, but also treated through a minimally invasive access, i.e. that in particular also the implant can be inserted through the access.

It is beneficial if the measuring instrument has a probe tip, and if an edge or a boundary of the defect is palpated with the probe tip for measurement of the defect. This method allows an experienced surgeon or arthroscopist to measure the defect without seeing it directly, or even if an inserted optical element displays a distorted image of the defect. The boundary line is then recorded by the detection device, so that the specific defect data file contains all the necessary data to prepare an implant corresponding to the defect.

It is beneficial if the defect is measured without contact. This has the advantage that the defect is not enlarged further by the measurement. For example, an electromagnetic radiation, in particular visible light, or ultrasound can be used for measurement of the defect.

To enable a surgeon to receive an even better impression of the defect, it is advantageous if the measuring instrument has an optical imaging unit, and if at least one defect image of the defect is recorded with the imaging unit. Thus, a surgeon can also see the defect directly in real time and assess it with an appropriate optical system, especially as the defect can thus be measured without contact, e.g. by evaluation of the defect image.

It is beneficial if an image data file of the at least one defect image is created by the imaging unit, and if the image data file is processed to form the defect data file. The data provided by the imaging unit can be processed in a simple manner to form the defect data file, so that all geometric data of the defect are known.

The defect data file can be obtained particularly quickly if the image data file is processed with a data processing device to form the defect data file.

In principle, a camera optimised for visible light, for example, with or without additional optical elements could be used as imaging unit. Alternatively, imaging units identifying or detecting microwave radiation, imaging units for optical coherence tomography (OCT) as well as ultrasonic imaging units could advantageously also be used. Advantageously, infrared sensors are used as imaging unit. These have the advantage that an illumination source is not absolutely necessary for image capture. As a result, a particularly small minimally invasive access is also sufficient to determine the defect data file.

The implant can be prepared advantageously, if a work tool is provided for machining the implant material, and if the implant material is machined with the work tool using the defect data file. When machining the implant material the defect data file is used, i.e. the implant is not prepared independently of the provided defect data file, but with specific consideration thereof.

In this case, it can be advantageous if the work tool has a reference element detectable by the detection device, and if the implant material is machined with the work tool with simultaneous detection of the position and orientation of the work tool. In this way, machining of the implant material can not only be monitored and controlled directly or indirectly, but can also be guided with the navigation system. For example, it would be conceivable to display a desired curve to be covered by the work tool together with the simultaneously determined actual path of movement of the work tool on a display device. A person preparing the implant can then recognise immediately how the work tool should be guided to prepare the implant in the desired manner.

A marking stylus is advantageously provided as the work tool for transferring the defect data file to the implant material. For example, a sheet implant material can be provided with the contour of the defect using a marking stylus, so that the implant can be separated from the implant material in a desired manner after marking or marking out the implant material.

According to a preferred variant of the method according to the invention, it can be arranged that a cutting device is provided as the work tool for cutting the support out of the implant material using the defect data file. In combination with a marking stylus or even without a marking stylus, the implant material can be prepared directly with the cutting device so that an implant of desired shape and size is formed.

In principle, it would be conceivable to have the work tool operated manually by an operator and thus prepare the implant. To enable the implant to be prepared fully automatically, it is beneficial if a machining device is provided for machining the implant material, if the defect data file is transferred to the machining device, and if the implant material is machined with the machining device. For example, the machining device can be a milling or cutting machine or a punch, which comprises a data processing unit, and the defect data file can be used to actuate the machining device in order to prepare an implant of the desired shape and size.

It is advantageous if the provided machining device comprises the work tool. It is then not absolutely necessary to navigate the work tool. However, a navigated work tool can be advantageous to prevent errors in the preparation of the implant.

The following description of preferred embodiments of the invention serves for more detailed explanation in association with the drawing:

### BRIEF DESCRIPTION OF THE DRAWINGS

FIG. 1 shows a first variant of a surgical system with a measuring instrument with probe tip;

FIG. 2 shows a second embodiment of a surgical system with a measuring instrument with imaging unit;

FIG. 3 shows a surgical system with a work tool for marking an implant material;

FIG. 4 shows a surgical system with a navigated work tool for separating a support or a replacement implant from a support material, and

FIG. 5 is a schematic representation of the method according to the invention.

### DETAILED DESCRIPTION OF THE DRAWINGS

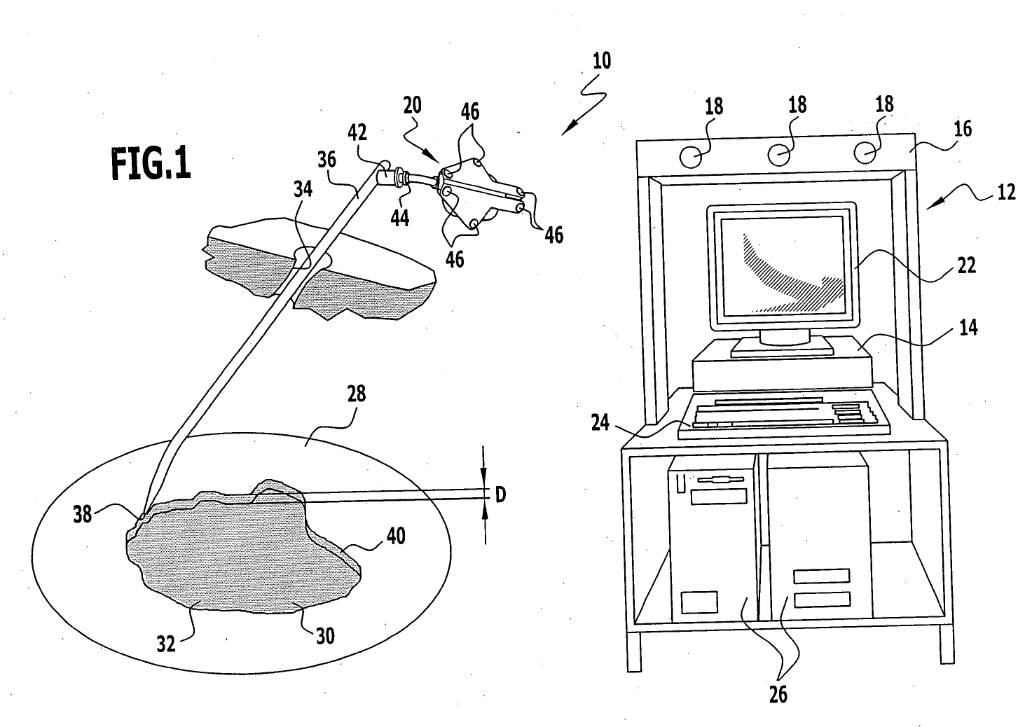

A surgical system given the overall reference 10 is shown in FIG. 1, which comprises a navigation system given the overall reference 12.

The navigation system 12 has a transmitting and receiving station 16, which is controlled by a computer 14 used as a data processing unit and comprises several transmitting and receiving units 18 for transmitting and receiving electromagnetic radiation, both in the visible and in the infrared range, or ultrasound, which is emitted or reflected by a reference element 20. The surgical system 10 and/or the navigation system 12 additionally comprise a display device in the form of a screen 22 as well as an input device in the form of a keyboard 24. To increase the efficiency of the system or systems 10 and 12, further arithmetic units 26 can co-operate with the computer 14. To assure the function of the navigation system 12 in optimum manner, at least three spatially separated transmitting and receiving units 18 are provided.

To treat, in particular fill, a defect in a bone and/or a cartilage of a joint of a human or animal body caused as a result of wear and/or a surgical procedure, the defect is measured by means of the surgical system. As a possible case of treatment, a section from a joint cartilage 28 of a human or animal joint (not shown in more detail) is provided with the reference 28. The joint cartilage 28 is damaged as a result of wear, an accident or a surgical procedure and therefore has a cartilage defect 30, which generally has a thickness D corresponding to a thickness of the cartilage layer. The thickness D can be constant or substantially constant over the entire cartilage defect 30. However, it can also differ slightly depending on a natural thickness of the joint cartilage 28. In particularly critical cases, the joint cartilage 28 is damaged down to the subchondral plate 32 lying beneath it.

The cartilage defect 30 is measured in order to determine in particular its size, shape, area and/or volume. For this purpose, a minimally invasive access 34, which is indicated schematically in FIG. 1, is opened in the human or animal body. The cartilage defect 30 is measured using a probe hook 36 serving as measuring instrument. A distal end of the probe hook 36 is configured in the form of a probe tip 38, with which an edge 40 or other boundary of the cartilage defect 30 can be scanned or palpated. Arranged at a proximal end of the probe hook 36 is a coupling stem 42, via which the probe hook 36 can be connected to a reference element 20, which has a coupling element 44 corresponding to the coupling stem 42. The reference element 20 has at least three passive or active position elements 46. Passive position elements 46 are suitable for reflecting and also the delayed radiation of electromagnetic radiation, which is emitted by the transmitting and receiving units 18. Conversely, active position elements 46 can themselves generate and emit electromagnetic radiation, which can be received by the transmitting and receiving units 18. If only active position elements 46 are used, pure receiving units can also be provided instead of the transmitting and receiving units 18.

The spatial position of the position elements 46 can be detected in real time by the navigation system 12. Since, moreover, the position of the position elements 46 relative to the probe hook 36, in particular to the probe tip 38, is known, the spatial position and the orientation of the probe hook 36 in space can be determined from the determined position of the position elements 46. Therefore, if the edge 40 of the cartilage defect 30 is palpated with the probe tip 38 of the probe hook 36, then a defect data file can be recorded by the navigation system 12, which contains data specifying the spatial position of the edge 40 in the area. Then, defect data specifying in particular the shape, size, area and/or volume of the cartilage defect 30 can be determined from this defect data file, for example, with the computer 14 and/or the arithmetic units 26.

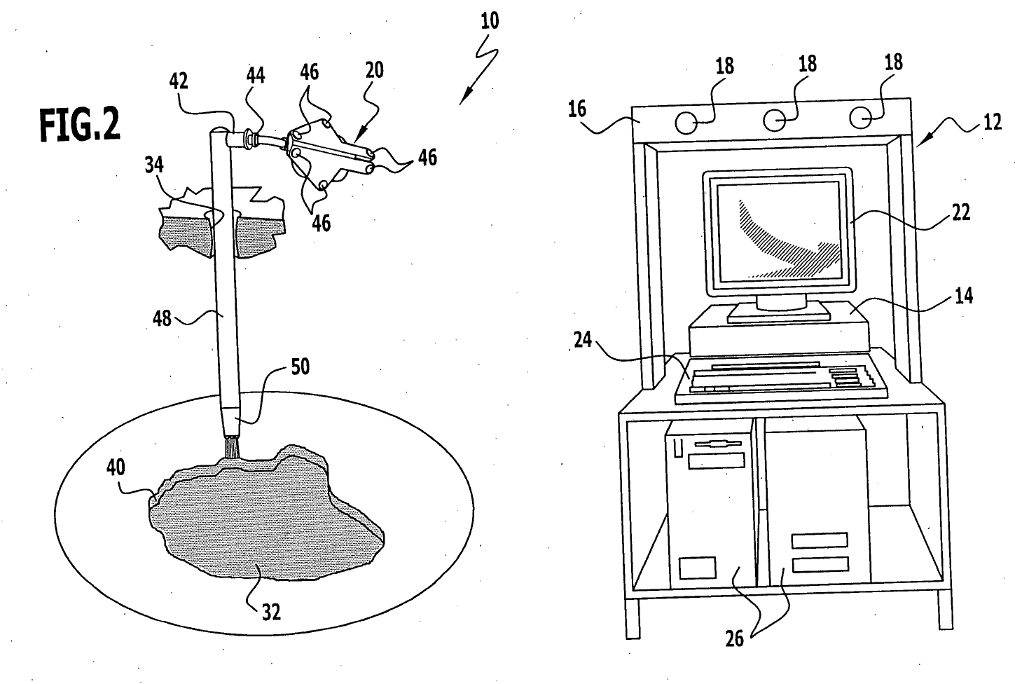

Alternatively to the probe hook 36, a measuring instrument 48 can also be used, as is shown schematically in FIG. 2. It has an imaging unit 50 arranged at its distal end, which comprises one or more infrared sensors, for example. The imaging unit could also be formed by an optical waveguide with an entrance window at the distal end of the instrument and a camera, which can be connected to the proximal end of the instrument or is arranged directly on the distal end of the instrument. The measuring instrument 48 can likewise be connected to the coupling element 44 of the reference element 20 via a coupling stem 42. The measuring instrument 48 can also be inserted into the human or animal body via a minimally invasive access 34. The cartilage defect 30 can be measured optically, i.e. without contact, by means of the imaging unit 50. For this the imaging unit 50 is moved over the cartilage defect 30 and quasi-scanned. Since the measuring instrument 48, just as the probe hook 36, is navigated during measurement of the cartilage defect 30, i.e. its position and orientation are detected by the navigation system 12, the spatial position and orientation of the cartilage defect 30 can be associated with each individual defect image or line scan of said defect recorded by the imaging unit 50. Thus, an image data file, which can be processed with the computer 14 and/or the arithmetic units 26 to form a defect data file, can be determined with the computer 14 and/or the arithmetic units 26 from a multiplicity of individual images or line scans of the cartilage defect 30 recorded with the imaging unit 50. In particular, the defect data file can also specify how deep the defect is, i.e. whether the defect only affects the cartilage or also the subchondral plate located beneath it or even the bone. If the latter is the case, then before insertion of the implant the defect on the subchondral plate and/or on the bone can be filled out with bone chips or bone replacement material so as not to put at risk the success of the treatment of the cartilage defect. Ideally, the defect data files obtained with the probe hook 36 and with the measuring instrument 48 are identical.

The surgical system 10 additionally comprises a work tool in the form of a marking stylus 52, which can be used to mark the contour 56 of the implant 58 to be prepared on an implant material 54, e.g. a collagen nonwoven. For this purpose, the marking stylus 52 has a tip 60, with which the implant material 54 can be marked out, i.e. provided with a line or the like, or marked in some other way, depending on the type of implant material, e.g. by providing a scratch trace. The implant material 54 preferably has a thickness D₁, which corresponds to the thickness D of the cartilage defect 30. The marked out implant material 54 can be further processed after marking, for example, with a cutting tool to separate the implant 58. This corresponds in shape, size, contour and volume to the cartilage defect 30.

For marking the implant material 54, the marking stylus 52 can be connected to a reference element 20, preferably via a coupling stem 52 arranged on the proximal end of the marking stylus 52 and a corresponding coupling element of the reference element 20. As a result, a movement of the tip 60 of the marking stylus 52 is navigable with the navigation system 12. In particular it is possible to represent a desired contour 62, which should be tracked with the marking stylus 52, on the screen 22. For control, an actual contour or actual movement path 64 of the tip 60 is simultaneously output on the screen together with the desired contour 62. A person, who is to mark the implant material 54 for further machining, then immediately recognises how the marking stylus 52 should be moved further.

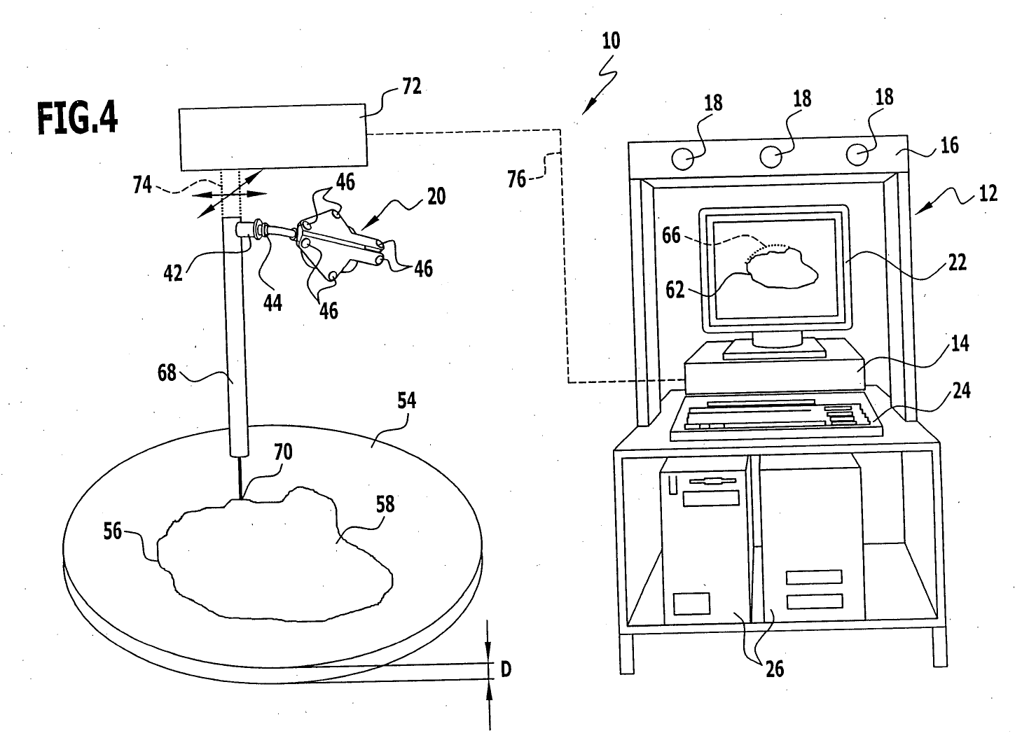

It will be described in more detail below how the implant 58 can be alternatively prepared from an implant material 54, in association with FIG. 4.

For this, a cutting tool 68, which is provided with a blade or cutting tip 70 on its distal end suitable for machining the implant material, is used instead of the marking stylus 52. Alternatively, the cutting tool could also be an electrically operable tool using a current or light, e.g. a laser, for cutting. The cutting tool 68 is also provided with a coupling stem 42, which is configured in a corresponding manner to the coupling stem 42 of the reference element 20.

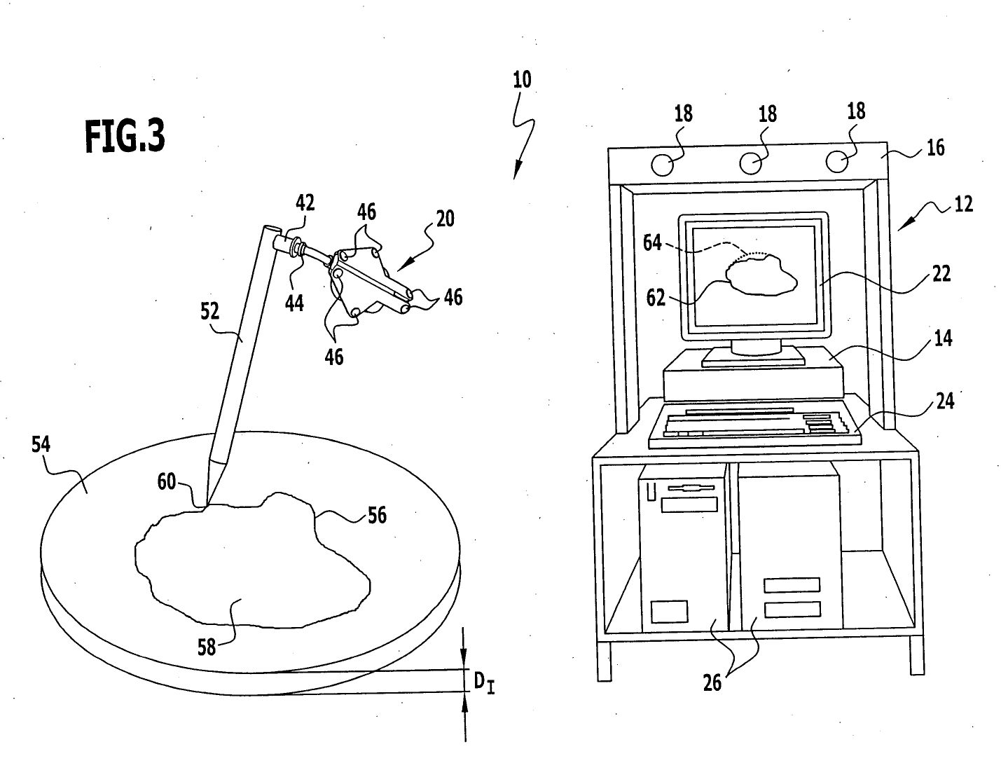

This can be used to navigate a movement of the cutting tool 68, if desired, as already in association with FIG. 3. If the desired contour 62 of the implant 58 is displayed on the screen 22, then an actual cutting path 66 of the cutting tip 70 can be simultaneously displayed together with the desired contour 62 to a person machining the implant material 54. The person conducting the preparation can then set the actual cutting path 66 in such a way that it is as identical as possible to the desired contour 62.

However, the implant material 54 cannot only be worked manually, as described above, but also by machine, i.e. with a machining device 72 shown schematically in FIG. 4. The machining device 72 can be coupled, for example, to the cutting tool 68 via a connecting unit 74 drawing in broken lines in FIG. 4, so that the cutting tool 68 can be moved beyond the implant material 54 in any desired direction, indicated by the directional arrow in FIG. 4. The machining device 72 can be actuated by the computer 14, for example. For this purpose, the computer 14 is connected to the machining device 72 via a control line, which is drawn in broken lines in FIG. 4. The advantage with electrically operable tools using a current or light, e.g. a laser, for cutting is that a supply of energy to the cutting tool can be interrupted immediately, and thus the cutting process can be stopped if the actual cutting path 66 deviates from the desired contour 62.

It is not absolutely essential to navigate the cutting tool 78 when using a machining device 72. Rather, it is sufficient to use the defect data file. For this it can be provided in particular that the defect data file is processed into a machining data file. Either the machining data file or the defect data file can be used to control the machining device 72.

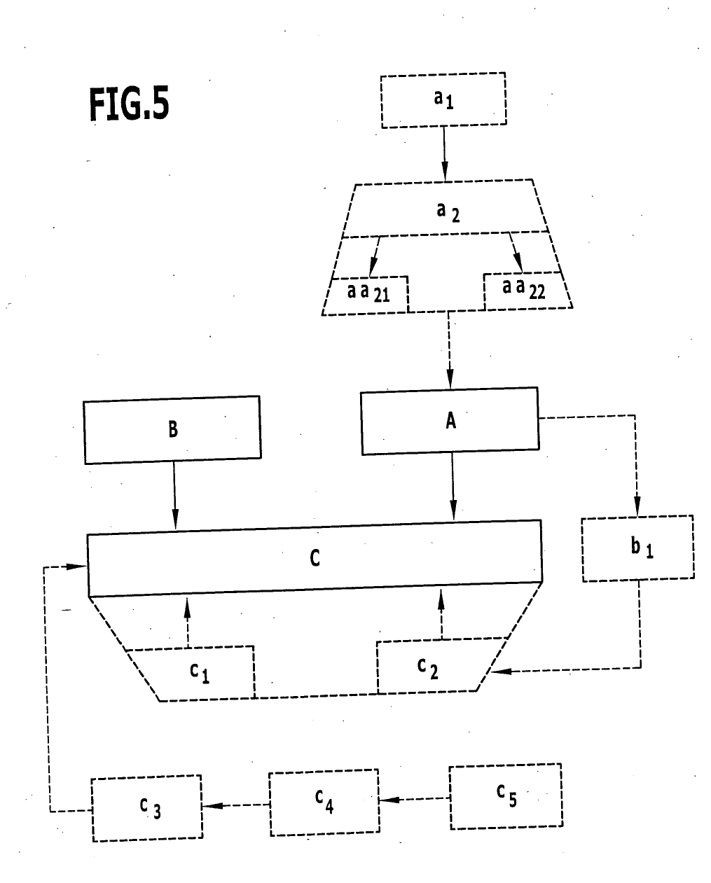

The method according to the invention for preparing an implant is shown schematically in FIG. 5. The method steps designated with capital and small letters are described below. Essential method steps are:
providing a data file, which contains data for description in particular of the shape, area and/or volume of the defect (A),
providing at least one implant material (B), and
preparing the implant from the implant material corresponding to the defect in shape and size using the defect data file (C).

The method steps (A) to (C) are essential for the method according to the invention.

For providing the defect data file the procedure can in particular be as follows:
opening a minimally invasive access to the human or animal body (a₁),
preparation of an edge of the defect, in particular removal of loose fragments not connected to the subchondral bone plate, or cutting an edge of the defect,
determination of the defect data file with a navigated measuring instrument (a₂),
wherein the defect data file can be determined without contact with an imaging unit (aa₂₁) or by probing with a probe instrument (aa₂₂).

The preparation of the implant can be performed by means of a navigated work tool (c₁) or with a machining device (c₂), wherein the machining device can be controlled using the defect data file. For this, the defect data file can be processed with a data processing unit to form a preparation data file (b₁), which is suitable for use to control the machining device.

In addition to the pure shaping preparation of the implant, cartilage cells from the body itself can be removed beforehand from the patient through a minimally invasive access (C₃). The removed body cells can be propagated in the laboratory until a required implant volume of the cartilage cells from the body itself is available (C₄). The propagated cartilage cells from the body itself can then be used to inoculate the cartilage replacement implant (c₅). The use of the method according to the invention and also of the surgical system according to the invention is not limited to the treatment of cartilage defects. Defects on the bone or other parts of the human body composed substantially of harder tissue can also be filled with an implant in the manner described.

When the implant material 54 is known, which preferably has cavities to receive cartilage cells from the body itself propagated in the laboratory, a cavity volume to be filled by the cartilage cells can be calculated from the determined volume of the cartilage defect 30. When the cavity volume is known, the propagation of the cartilage cells can be conducted in an optimised manner in the laboratory, i.e. the number of cartilage cells to be cultured needs only to be sufficient to allow the implant 58 to be adequately inoculated with cartilage cells.

What is claimed is:

1. Surgical system for the preparation of an implant for filling a defect on a bone and/or a cartilage of a human or animal body formed as a result of a trauma and/or degeneration, wherein the system enables a provided defect data file to be processed, which contains data in particular for the description of shape, area, height and/or volume of the defect, and wherein the system enables the implant corresponding to the defect in shape and size to be prepared from at least one provided implant material using the defect data file.

2. System according to claim 1, wherein the implant is a cartilage replacement implant for autologous chondrocyte transplantation (ACT), and the defect forming a cartilage defect can be filled with the cartilage replacement implant.

3. System according to claim 2, wherein the implant material is a support material suitable for inoculation with cartilage cells, the cartilage replacement implant comprises a support for the cartilage cells, and the support is prepared from the support material.

4. System according to claim 3, wherein the support material is a nonwoven material.

5. System according to claim 3, wherein the support can be inoculated with cartilage cells.

6. System according to claim 5, wherein the support can be inoculated with cartilage cells, which are cartilage cells from the body itself propagated in the laboratory, which were removed from the human or animal body before insertion of the cartilage replacement implant.

7. System according to claim 3, wherein the defect volume of the cartilage defect can be determined from the defect data file, and a provided implant volume of cartilage cells is sufficient to adequately inoculate the support with cartilage cells for implantation of the cartilage replacement implant.

8. System according to claim 7, wherein the provided support material has cavities to receive cartilage cells, the cavities of the support have a cavity volume, and a provided implantation volume corresponds to the cavity volume or approximately to the cavity volume of the support.

9. System according to claim 1, wherein a data processing unit is provided for processing the defect data file with the data processing unit to form a preparation data file, wherein the preparation data file contains data in particular for the description of shape, area, height and/or volume of the implant to be prepared.

10. System according to claim 1, wherein a surgical instrument is provided for opening a minimally invasive access into the human or animal body to determine the defect data file.

11. System according to claim 1, wherein a navigation system is provided with a detection device, a measuring instrument is provided, on which a reference element detectable by the detection device of the navigation system can be arranged for determination of the position and orientation of the measuring instrument in space, and the defect can be measured with the measuring instrument for determination of the defect data file and at the same time the position and orientation of the measuring instrument can be determined in space with the navigation system.

12. System according to claim 11, wherein the measuring instrument can be inserted into the human or animal body through a minimally invasive access.

13. System according to claim 11, wherein the measuring instrument has a probe tip, and an edge or a boundary of the defect can be palpated with the probe tip for measurement of the defect.

14. System according to claim 11, wherein the measuring instrument is configured such that the defect can be measured without contact.

15. System according to claim 11, wherein the measuring instrument has an optical imaging unit, and at least one defect image of the defect can be recorded with the imaging unit.

16. System according to claim 15, wherein an image data file of the at least one defect image can be created by the imaging unit, and the image data file can be processed to form the defect data file.

17. System according to claim 16, wherein a data processing device is provided for processing the image data file with the data processing unit to form the defect data file.

18. System according to claim 15, wherein at least one infrared sensor is provided as the imaging unit.

19. System according to claim 1, wherein a work tool is provided for machining the implant material, and the implant material can be machined with the work tool using the defect data file.

20. System according to claim 11, wherein a work tool is provided for machining the implant material, and the implant material can be machined with the work tool using the defect data file.

21. System according to claim 20, wherein the work tool has a reference element detectable by the detection device, and the implant material can be machined with the work tool with simultaneous detection of the position and orientation of the work tool.

22. System according to claim 19, wherein the work tool comprises a marking tool for transferring the defect data file to the implant material.

23. System according to claim 19, wherein the work tool comprises a cutting device for cutting the support out of the implant material using the defect data file.

24. System according to claim 19, wherein a machining device is provided for machining the implant material, that the defect data file is transferable to the machining device, and that the implant material can be machined with the machining device.

25. System according to claim 24, wherein the machining device comprises the work tool.

26. System according to claim 1, wherein the system comprises the implant.

27. Method for preparing an implant for filling a defect on a bone and/or a cartilage of a human or animal body present as a result of a trauma and/or degeneration, comprising the steps:
providing at least one implant material;
providing a defect data file, which contains data for the description in particular of shape, area and/or volume of the defect, and
preparing the implant from the implant material corresponding to the defect in shape and size using the defect data file.

28. Method according to claim 27, wherein the defect is a cartilage defect, the implant is a cartilage replacement implant for autologous chondrocyte transplantation (ACT), and the cartilage defect is filled with the cartilage replacement implant.

29. Method according to claim 28, wherein a support material suitable for inoculation with cartilage cells is provided as implant material, the cartilage replacement implant comprises a support for the cartilage cells, and the support is prepared from the support material.

30. Method according to claim 29, wherein a nonwoven material is used as support material.

31. Method according to claim 29, wherein the support is inoculated with cartilage cells after preparation and before implantation.

32. Method according to claims 29, wherein cartilage cells are removed from the human or animal body before implantation of the cartilage replacement implant, the removed cartilage cells from the body itself are propagated in the laboratory, and the propagated autologous cartilage cells are provided for inoculation of the support.

33. Method according to claim 29, wherein the defect volume of the cartilage defect is determined from the defect data file, and that an implant volume of cartilage cells is provided, wherein the implantation volume is sufficient to adequately inoculate the support with cartilage cells for an implantation of the cartilage replacement implant.

34. Method according to claim 33, wherein the support material provided has cavities to receive cartilage cells, the cavities of the support have a cavity volume, and an implantation volume is provided, which corresponds to the cavity volume or approximately to the cavity volume of the support.

35. Method according to claim 27, wherein the defect data file is processed with a data processing unit to form a preparation data file, wherein the preparation data file contains data for the description in particular of shape, area and/or volume of the implant to be prepared.

36. Method according to claim 27, wherein a minimally invasive access into the human or animal body is opened to determine the defect data file.

37. Method according to claim 27, wherein an edge of the defect is prepared before determination of the defect data file.

38. Method according to claim 27, wherein a measuring instrument is provided, on which a reference element detectable by a detection device of a navigation system can be arranged for determination of the position and orientation of the measuring instrument in space, and the defect is measured with the measuring instrument for determination of the defect data file and at the same time the position and orientation of the measuring instrument is determined in space.

39. Method according to claim 38, wherein the measuring instrument is inserted into the human or animal body through the minimally invasive access.

40. Method according to claim 38, wherein the measuring instrument has a probe tip, and an edge or a boundary of the defect is palpated with the probe tip for measurement of the defect.

41. Method according to claim 27, wherein the defect is measured without contact.

42. Method according to claim 27, wherein the measuring instrument has an optical imaging unit, and at least one defect image of the defect is recorded with the imaging unit.

43. Method according to claim 42, wherein an image data file of the at least one defect image is provided by the imaging unit, and the image data file is processed to form the defect data file.

44. Method according to claim 43, wherein the image data file is processed with a data processing device to form the defect data file.

45. Method according to claim 42, wherein infrared sensors are used as the imaging unit.

46. Method according to claim 27, wherein a work tool is provided for machining the implant material, and the implant material is machined with the work tool using the defect data file.

47. Method according to claim 38, wherein a work tool is provided for machining the implant material, and the implant material is machined with the work tool using the defect data file.

48. Method according to claim 47, wherein the work tool has a reference element detectable by the detection device, and the implant material is machined with the work tool with simultaneous detection of the position and orientation of the work tool.

49. Method according to claim 46, wherein a marking stylus is provided as the work tool for transferring the defect data file to the implant material.

50. Method according to claim 46, wherein a cutting device is provided as the work tool for cutting the support out of the implant material using the defect data file.

51. Method according to claim 46, wherein a machining device is provided for machining the implant material, the defect data file is transferred to the machining device, and the implant material is machined with the machining device.

52. Method according to claim 51, wherein the provided machining device comprises the work tool.
